(19) United States
(12) Patent Application Publication
(10) Pub. No.: US 2026/0325016 A1
(43) Pub. Date: Oct. 22, 2026

(54) SYSTEM FOR BLOOD GLUCOSE MANAGEMENT USING CONTINUOUS GLUCOSE MONITOR AND METHOD THEREOF

(71) Applicant: ODN Co., Ltd., Seoul (KR)

(72) Inventors: Sang Youl RHEE, Seoul (KR); Hyun Jung PARK, Seoul (KR); Tae Hyun PARK, Seoul (KR); Seung Cheol AHN, Seoul (KR)

(21) Appl. No.: 19/648,501

(22) Filed: Apr. 15, 2026

(30) Foreign Application Priority Data
Apr. 17, 2025 (KR) 10-2025-0050269

(51) Int. Cl. A61B 5/145; A61B 5/00; G16H 10/60; G16H 50/20; G16H 50/30

(52) U.S. Cl. CPC A61B 5/14532; A61B 5/6804; A61B 5/7275; A61B 5/7435; A61B 5/746; G16H 10/60; G16H 50/20; G16H 50/30

(57) ABSTRACT

Provided are a blood glucose management system using a continuous glucose monitor and a method therefor. The blood glucose management system may include a data collector configured to collect blood glucose data measured through a continuous glucose monitor worn by a patient; a data analyzer configured to analyze the collected blood glucose data to calculate values of a plurality of blood glucose variables representing a blood glucose state of the patient; and a data provider configured to provide the values of the blood glucose variables through a monitoring board. By using the system, the blood glucose states of patients can be thoroughly monitored and managed.

### STATEMENT REGARDING SPONSORED RESEARCH OR DEVELOPMENT

The present disclosure was made with the support of the Ministry of Trade, Industry and Energy (MOTIE) of the Republic of Korea, under a program supervised by the Korea Institute for Advancement of Technology (KIAT), under Project Unique Number 2410013887 and Project Number 07892971, entitled “Industrial Technology Development Program for the Bio Industry (R&D),” specifically the project titled “Development of an AI-based Biosignal Monitoring and Real-time Early Warning System for Severe Trauma Patients, Beacon (Biosignal Emergency Advanced Critical Observation Network),” performed by ODN Co., Ltd. during the period from Jan. 1, 2026 to Dec. 31, 2026.

### CROSS-REFERENCE TO RELATED APPLICATION

This application claims priority to Korean Patent Application No. 10-2025-0050269, filed on Apr. 17, 2025, in the Ministry of Intellectual Property (MOIP) of the Republic of Korea, the disclosure of which is incorporated herein by reference.

### BACKGROUND

### Field of the Disclosure

The present disclosure relates to a technology for monitoring and managing blood glucose of a patient using a continuous glucose monitor (CGM).

### Description of the Related Art

Blood glucose is one of the core biomarkers representing a metabolic state of a human body, and plays a very important role in diagnosis, prognosis evaluation, and treatment progress observation of various metabolic diseases including diabetes. In particular, since blood glucose abnormalities such as hypoglycemia or hyperglycemia can cause acute or chronic complications, stably managing blood glucose is a common task given to both patients and medical staff.

Recently, a continuous glucose monitor (CGM) has been widely used to measure a blood glucose state of a patient in real time. The CGM automatically measures blood glucose levels at regular intervals through a sensor and transmits them to a mobile terminal or an external device through wireless communication, thereby being able to provide much more detailed and continuous data than an existing self-monitoring blood glucose meter. However, there still exist some practical difficulties in blood glucose management using such a CGM.

First, the blood glucose data provided by the CGM is mostly readable only in a dedicated app of a manufacturer of the device, and has low compatibility with healthcare apps of other companies. Due to this, it is difficult to integrally manage or analyze the blood glucose data within a daily healthcare platform used by the patient.

Second, since the CGM is generally not linked with a legacy system, such as an electronic medical record (EMR), of a medical institution, it is difficult to share meaningful blood glucose data collected by the patient with medical staff in real time. Accordingly, the clinical utilization possibility of the blood glucose data can be hindered, and the decision-making of the medical staff can also be delayed. Furthermore, due to the closed nature, structural limitations, and complexity of the legacy system, it is also practically very difficult to integrate an analysis and monitoring function of the blood glucose data into the corresponding system.

Therefore, so as to maximize the effects of blood glucose management using the CGM, there is a need to develop a system capable of providing an analysis and monitoring function for the blood glucose data of a patient and, at the same time, supporting interoperability between the CGM and a legacy system of a medical institution or a healthcare platform.

### SUMMARY

Therefore, the present disclosure has been made in view of the above problems, and it is an object of the present disclosure to provide a system capable of effectively managing a blood glucose of a patient using a continuous glucose monitor (CGM), and a method thereof.

Specifically, a technical problem to be solved by some embodiments of the present disclosure is to provide a system capable of accurately analyzing and monitoring blood glucose data of the patient collected through the CGM, and a method thereof.

It is another object of the present disclosure to provide a system capable of early predicting an occurrence of a blood glucose abnormality in the patient based on the blood glucose data collected through the CGM, and supporting a preemptive management measure, and a method thereof.

It is yet another object of the present disclosure to provide a system capable of supporting interoperability between the CGM and a legacy system of a medical institution or a health management platform, and a method thereof.

The technical problems of the present disclosure are not limited to the above-mentioned technical problems, and other technical problems not mentioned will be clearly understood by those skilled in the art to which the present disclosure pertains from the following description.

In accordance with an aspect of the present disclosure, the above and other objects can be accomplished by the provision of a system for blood glucose management, the system including: a data collector configured to collect blood glucose data of a patient, wherein the blood glucose data is measured through a continuous glucose monitor worn by the patient; a data analyzer configured to analyze the collected blood glucose data to calculate values of a plurality of blood glucose variables representing a blood glucose state of the patient; and a data provider configured to provide the values of the blood glucose variables through a monitoring board. Here, the blood glucose management system may be linked with a legacy system of a medical institution, and may be configured to perform processing based on the blood glucose data independently of the legacy system.

In some embodiments, the legacy system may include an Electronic Medical Record (EMR) system.

In some embodiments, the blood glucose management system may provide at least some of the values of the blood glucose variables to the legacy system, and receive medical information of the patient from the legacy system.

In some embodiments, the data collector may collect blood glucose data of each of a plurality of patients, and the data provider may integrally provide blood glucose-related information of the patients through the monitoring board.

In some embodiments, the data collector may collect the blood glucose data from a manufacturer server of the continuous glucose monitor through an Application Programming Interface (API).

In some embodiments, the blood glucose variables may include a Time In Tight Range (TITR), wherein the TITR means a ratio of time during which a blood glucose is within a predetermined range, wherein an upper limit of the predetermined range is set to a value between 130 mg/dL and 150 mg/dL, and a lower limit of the predetermined range is set to a value between 60 mg/dL and 90 mg/dL.

In some embodiments, the blood glucose variables may include a Glycemia Risk Index (GRI), wherein the GRI is calculated based on a hypoglycemia risk index and a hyperglycemia risk index, wherein a higher weight is assigned to the hypoglycemia risk index than to the hyperglycemia risk index.

In some embodiments, the data analyzer may acquire clinical information of the patient from the legacy system, and predict a risk level of an occurrence of a blood glucose abnormality in the patient within a specific time by jointly analyzing the collected blood glucose data and the clinical information through a machine learning model, the clinical information may include at least one of demographic information, diagnosis information, prescription information, a medication history, and a disease history of the patient, and the blood glucose management system may further include a notifier configured to provide a notification based on the predicted risk level.

In some embodiments, the machine learning model may include: an encoder configured to generate variable embeddings corresponding to the blood glucose variables; an interaction encoder configured to generate interaction embeddings corresponding to predefined blood glucose variable pairs based on the variable embeddings; a variable weight estimator configured to estimate a variable weight, specialized for the patient, for each of the blood glucose variables based on the clinical information; an interaction weight estimator configured to estimate an interaction weight, specialized for the patient, for each of the blood glucose variable pairs based on the clinical information; an aggregator configured to generate an integrated embedding by aggregating the variable embeddings, in which the variable weight is reflected, and the interaction embeddings in which the interaction weight is reflected; and a predictor configured to predict the risk level based on the integrated embedding.

In some embodiments, the notifier may: withhold providing the notification until a preset standby timer expires when the predicted risk level is equal to or greater than a threshold value; immediately provide the notification when an event increasing the occurrence risk of the blood glucose abnormality is detected in the patient before the standby timer expires; and reset the standby timer and predicts the risk level again through the machine learning model, when an event decreasing the occurrence risk of the blood glucose abnormality is detected in the patient before the standby timer expires.

In some embodiments, the monitoring board may include: a first interface displaying the values of the blood glucose variables; a second interface displaying blood glucose-related information of the patient by time section; a third interface displaying a ratio of time during which the blood glucose of the patient is maintained within a target blood glucose range; and a fourth interface displaying a daily blood glucose profile of the patient.

In some embodiments, the blood glucose management system may include a Backend For Frontend (BFF) server, a Fast Healthcare Interoperability Resource (FHIR) relay server, a gateway server, an FHIR server, and an internal integration server, the data collector, the data analyzer, and the data provider may be implemented in the internal integration server, the FHIR server and the internal integration server may be located together with the legacy system in an internal network of the medical institution, the BFF server may be configured to transfer health data, received from a terminal of the patient, to the FHIR relay server, the FHIR relay server may be configured to convert the health data, received from the BFF server, according to an FHIR protocol and transfer the converted health data to the gateway server, the gateway server may be configured to transfer the converted health data to the FHIR server located in the internal network, and the FHIR server may be configured to transfer the converted health data to the internal integration server.

In accordance with another aspect of the present disclosure, provided is a method for blood glucose management using a continuous glucose monitor, the method being performed by at least one processor of a blood glucose management system and including: collecting blood glucose data measured through the continuous glucose monitor worn by a patient; calculating values of a plurality of blood glucose variables representing a blood glucose state of the patient by analyzing the collected blood glucose data; and providing the values of the blood glucose variables through a monitoring board. Here, the blood glucose management system may be linked with a legacy system of a medical institution, and may be configured to perform processing based on the blood glucose data independently of the legacy system.

In accordance with yet another aspect of the present disclosure, provided is a computer program stored in a computer-readable recording medium to execute, in combination with a processor of a computer: collecting blood glucose data measured through a continuous glucose monitor worn by a patient; calculating values of a plurality of blood glucose variables representing a blood glucose state of the patient by analyzing the collected blood glucose data; and providing the values of the blood glucose variables through a monitoring board.

### BRIEF DESCRIPTION OF THE DRAWINGS

The above and other objects, features and other advantages of the present disclosure will be more clearly understood from the following detailed description taken in conjunction with the accompanying drawings, in which:

FIGS. 1 and 2 are views for explaining an exemplary operating environment of a blood glucose management system according to some embodiments of the present disclosure;

FIG. 3 is an exemplary block diagram for describing the configuration and operation of the blood glucose management system according to some embodiments of the present disclosure;

FIG. 4 is an exemplary view illustrating an implementation example of the blood glucose management system according to some embodiments of the present disclosure;

FIG. 5 is an exemplary flowchart illustrating a blood glucose management method using a continuous glucose monitor (CGM) according to some embodiments of the present disclosure;

FIG. 6 is an exemplary view for explaining a screen configuration of a monitoring board according to some embodiments of the present disclosure;

FIGS. 7A to 7E illustrate implementation examples of the monitoring board;

FIG. 8 is an exemplary view illustrating input/output data of a machine learning model according to some embodiments of the present disclosure;

FIG. 9 is a view for explaining an exemplary structure and operation of the machine learning model according to some embodiments of the present disclosure;

FIG. 10 is an exemplary view for explaining a model training method based on multi-task learning according to some embodiments of the present disclosure; and

FIG. 11 illustrates an exemplary computing device capable of implementing the blood glucose management system according to some embodiments of the present disclosure.

### DETAILED DESCRIPTION

Hereinafter, various embodiments of the present disclosure will be described in detail with reference to the accompanying drawings. Advantages and features of the present disclosure and methods of achieving them will become apparent with reference to embodiments described below in detail in conjunction with the accompanying drawings. However, the technical spirit of the present disclosure is not limited to the following embodiments, but may be implemented in various different forms. The following embodiments are merely provided to make the technical spirit of the present disclosure complete and to fully inform those of ordinary skill in the art to which the present disclosure pertains of the scope of the present disclosure, and the technical spirit of the present disclosure is only defined by the scope of the claims.

When it is determined that a detailed description of a related known configuration or function may obscure the gist of the present disclosure in describing the various embodiments of the present disclosure, the detailed description thereof will be omitted.

Unless otherwise defined, terms (including technical and scientific terms) used in the following embodiments may be used with meanings commonly understood by those of ordinary skill in the art to which the present disclosure pertains, but this may vary depending on the intentions of technicians working in the related field, precedents, the emergence of new technologies, and the like. The terms used in the present disclosure are for describing the embodiments and are not intended to limit the scope of the present disclosure.

Singular expressions used in the following embodiments include plural concepts unless the context clearly specifies them as singular. In addition, the plural expressions include singular concepts unless the context clearly specifies them as plural.

In addition, terms such as first, second, A, B, (a), and (b) used in the following embodiments are merely used to distinguish one component from another component, and the essence, order, or sequence of the corresponding component is not limited by the terms.

Components described using terms such as “~unit”, “module”, “block”, and “~er” or “~or” in the following embodiments and functional blocks shown in the drawings may be implemented in the form of software, hardware, or a combination thereof. The software may include, for example, machine code, firmware, embedded code (or software), application software, or a combination thereof. In addition, the hardware may include, for example, an electrical circuit, an electronic circuit, a processor, a computer, an integrated circuit, integrated circuit cores, a passive element, or a combination thereof. As a more specific example, the “~unit”, the “module”, and the like may include components (such as software components, object-oriented software components, class components, and task components), processes, functions, properties, procedures, subroutines, segments of program code, drivers, firmware, microcode, circuitry, data, databases, data structures, tables, arrays, and variables.

Hereinafter, various embodiments of the present disclosure will be described in detail with reference to the accompanying drawings.

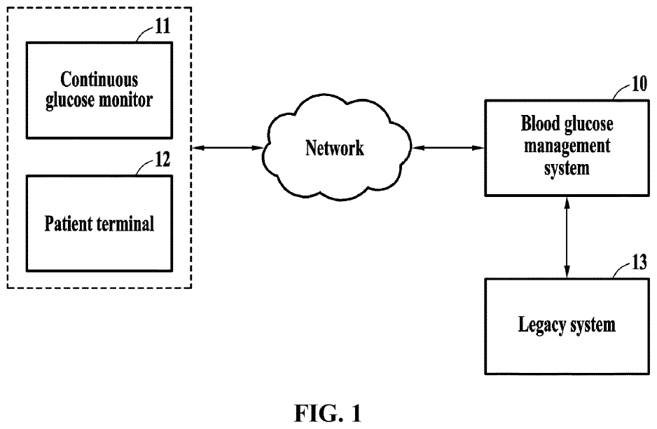

FIG. 1 is a view for explaining an exemplary operating environment of a blood glucose management system 10 according to some embodiments of the present disclosure.

As shown in FIG. 1, the blood glucose management system 10 according to embodiments is a computing device/system equipped with a blood glucose management function for one or more patients, and may operate in conjunction with a continuous glucose monitor (CGM) 11 and a legacy system 13 of a medical institution. However, the scope of the present disclosure is not limited thereto. Although FIG. 1 illustrates a case where the blood glucose management system 10 is linked with one CGM 11 as an example, the blood glucose management system 10 may be configured to be linked with a plurality of CGMs so as to integrally perform blood glucose management for a plurality of patients.

In the operating environment illustrated in FIG. 1, the blood glucose management system 10 may collect blood glucose data of a patient through the CGM 11, analyze the collected blood glucose data, and perform a blood glucose management function (e.g., provision of blood glucose-related information, notification of a blood glucose abnormality, etc.) for the patient. Here, the blood glucose data may be understood as continuous blood glucose data which is a kind of time-series data.

In addition, the blood glucose management system 10 may provide the collected blood glucose data and/or an analysis result thereof (e.g., at least a part of information indicating a blood glucose state) to a legacy system 13 of a medical institution. In this case, since the blood glucose management system 10 performs processing based on the blood glucose data independently of the legacy system 13, blood glucose-related information of the patient may be reflected (or integrated) into the legacy system 13 without affecting a structure or performance of the legacy system 13.

In addition, the blood glucose management system 10 may acquire medical information (e.g., various clinical information) of the patient from the legacy system 13, and perform patient-customized blood glucose management by jointly analyzing the blood glucose data and the medical information of the patient. For example, the blood glucose management system 10 may early predict a risk level of an occurrence of a blood glucose abnormality for the corresponding patient within a specific time (e.g., 24 hours, 6 hours, etc.) through a machine learning model, and preemptively provide a notification for blood glucose management to the patient based on this. Such a notification may be provided through an app (e.g., a healthcare app, etc.) installed in a patient terminal 12, but the scope of the present disclosure is not limited thereto. The blood glucose management system 10 may also provide such a notification to medical staff.

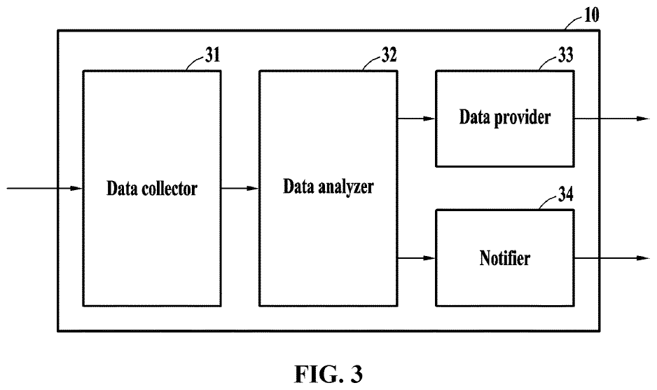

A more detailed description of the configuration and operation of the blood glucose management system 10 will be described below with reference to FIG. 3 and the subsequent drawings.

For reference, when the blood glucose management system 10 is implemented to further provide other health management services in addition to blood glucose management, the blood glucose management system 10 may be named as a ‘health management system’ or the like.

The CGM 11 is a device provided with a function that can continuously measure a blood glucose state of a patient at predetermined intervals through a sensor attached to the patient and transmit the measured blood glucose state to an external device (e.g., the patient terminal 12, etc.). The CGM 11 may be used to monitor the blood glucose state of the patient in real time or near real time, and unlike a traditional self-monitoring blood glucose meter, has an advantage that a blood glucose trend can be continuously grasped without a fingertip blood draw.

The patient terminal 12 means a patient-side device (that is, a computing device) that can be interlocked with the blood glucose management system 10 and the like. A healthcare app (e.g., a blood glucose management app, etc.) linked with the blood glucose management system 10 may be installed in the patient terminal 12, and the patient may be provided with a blood glucose management function through the app (e.g., being provided with a blood glucose abnormality notification, etc.). In addition, the patient terminal 12 may transfer the blood glucose data received from the CGM 11 to the blood glucose management system 10.

The patient terminal 12 may be implemented as various types of devices such as, for example, a smartphone, a tablet, a desktop, a smartwatch, or other wearable devices, but is not limited thereto.

The legacy system 13 means an information system already constructed and operated in a medical institution. The legacy system 13 may acquire blood glucose-related information (e.g., blood glucose data, blood glucose state information, etc.) of a patient from the blood glucose management system 10, and may also provide medical information of the patient to the blood glucose management system 10.

Examples of the legacy system 13 may include an Electronic Medical Record (EMR), a Hospital Information System (HIS), an Order Communication System (OCS), a Picture Archiving and Communication System (PACS), a Laboratory Information System (LIS), and the like, but are not limited thereto. Since the systems provided as examples are generally constructed based on a closed structure or a non-standard interface, direct interlocking with an external system is often limited.

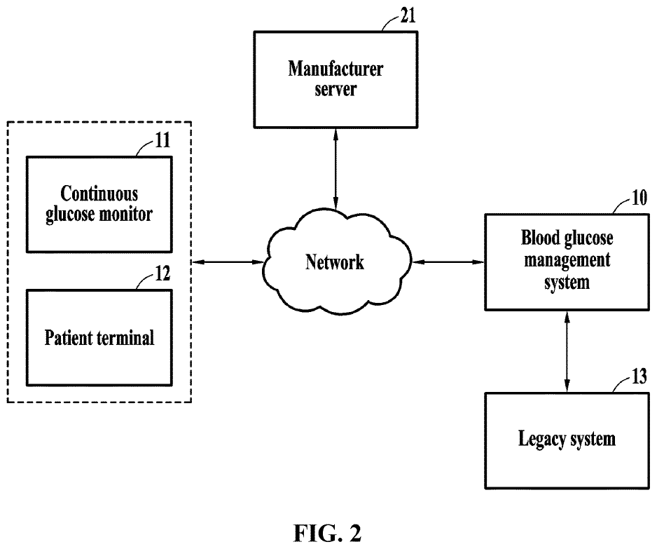

In some embodiments, the blood glucose management system 10 may further operate in conjunction with a manufacturer server 21 of the CGM 11, as illustrated in FIG. 2. The manufacturer server 21 is a server for managing the blood glucose data measured (or provided/generated) by the CGM 11, and may be implemented based on, for example, a cloud, but is not limited thereto. In the environment illustrated in FIG. 2, the blood glucose management system 10 may collect the blood glucose data of the patient, measured by the CGM 11, through an Application Programming Interface (API) provided by the manufacturer server 21. In this case, even in an environment where direct interlocking with the CGM 11 is limited (e.g., a case where the CGM 11 is a product of another manufacturer, etc.), the blood glucose data of the patient may be stably collected.

As illustrated in FIG. 1 or 2, the patient terminal 12, the blood glucose management system 10, the manufacturer server 21, and the like may communicate through a network. Here, the network may be implemented as all types of wired/wireless networks, such as a Local Area Network (LAN), a Wide Area Network (WAN), a mobile radio communication network, a Wireless Broadband Internet (Wibro), and the like.

As described above, the exemplary operating environment of the blood glucose management system 10 according to some embodiments of the present disclosure has been described with reference to FIGS. 1 and 2. Hereinafter, the configuration and operation of the blood glucose management system 10 described above will be described with reference to FIG. 3 and the subsequent drawings.

FIG. 3 is an exemplary block diagram for describing the configuration and operation of the blood glucose management system 10 according to some embodiments of the present disclosure.

As illustrated in FIG. 3, the blood glucose management system 10 according to embodiments may include a data collector 31, a data analyzer 32, a data provider 33, and a notifier 34. However, only components related to the embodiments of the present disclosure are illustrated in FIG. 3. Therefore, those of ordinary skill in the art to which the present disclosure pertains may understand that other general-purpose components (e.g., a processor, a memory, an input/output device, a database, etc.) may be further included in addition to the components 31 to 34 illustrated in FIG. 3.

In addition, the components 31 to 34 of the blood glucose management system 10 illustrated in FIG. 3 represent functionally divided functional elements, and a plurality of components may be implemented in a form of being integrated with each other in an actual physical environment, or a specific component may be implemented in a form of being separated into a plurality of sub-components. Hereinafter, each of the components 31 to 34 of the blood glucose management system 10 will be described.

The data collector 31 is a module that collects blood glucose data of a patient. For example, the data collector 31 may collect the blood glucose data through the CGM 11, and may also collect the blood glucose data from the manufacturer server 21.

Next, the data analyzer 32 is a module that analyzes the collected blood glucose data. The data analyzer 32 may derive various information necessary for blood glucose management by analyzing the blood glucose data.

For example, the data analyzer 32 may calculate values of various blood glucose variables, which represent a blood glucose state of the patient, by analyzing the blood glucose data. Examples of the blood glucose variables may include a Time in Range (TIR), a Time Above Range (TAR), a Time Below Range (TBR), a Time In Tight Range (TITR), a Glycemia Risk Index (GRI), an average blood glucose, a standard deviation, a Glucose Variability (CV), a Glucose Management Indicator (GMI), and the like, but are not limited thereto. For meanings of the illustrated blood glucose variables, a method of calculating variable values, and the like, Table 1 below may be referred to.

TABLE 1

| Blood glucose | |
|---|---|
| variable | Description |
| TIR | Ratio of time during which blood glucose is within a |
| | target range (e.g., 70 mg/dL to 180 mg/dL) |
| TAR | Ratio of time during which the blood glucose is in a |
| | state exceeding the target range (e.g., >180 mg/dL, >250 |
| | mg/dL) (i.e., a ratio of time spent in a hyperglycemia |
| | state or a ratio of time exposed to a hyperglycemia risk) |
| TBR | Ratio of time during which the blood glucose is in a state |
| | less than the target range (e.g., <70 mg/dL, <54 mg/dL) |
| | (i.e., a ratio of time spent in a hypoglycemia state or |
| | a ratio of time exposed to a hypoglycemia risk) |
| TITR | Ratio of time during which the blood glucose is within |
| | a stricter target range than the TIR (e.g., 70 mg/dL |
| | to 140 mg/dL) |
| GRI | Index integrally quantifying the hyperglycemia risk |
| | and the hypoglycemia risk |
| Blood glucose | Ratio obtained by dividing the standard deviation of |
| variable | blood glucose by the average blood glucose (i.e., |
| | relative variability) |
| GMI | Index similar to an HbA1c level estimated from |
| | the average blood glucose |

For reference, a target range of the TITR may be set such that, for example, an upper limit is set to a value between about 130 mg/dL and 150 mg/dL and a lower limit is set to a value between about 60 mg/dL and 90 mg/dL, and may be preferably set to a range of about 70 mg/dL to 140 mg/dL. In this case, it was confirmed that a blood glucose risk that may occur in the patient may be effectively managed. However, the scope of the present disclosure is not limited thereto, and a target blood glucose range related to the TIR and the TITR may be set differently according to a type, a characteristic, and the like of the patient.

In addition, the GRI may be calculated based on a hypoglycemia risk index and a hyperglycemia risk index. For example, the GRI may be calculated based on a weighted sum of the two risk indices, and in this case, a higher weight may be assigned to the hypoglycemia risk index than the hyperglycemia risk index. By doing so, the hypoglycemia risk may be more effectively determined. The hypoglycemia risk index and the hyperglycemia risk index may be calculated based on a ratio of time during which the blood glucose of the patient is in a state less than the target range and a ratio of time during which the blood glucose is in a state exceeding the target range, respectively, but the scope of the present disclosure is not limited thereto. As a more specific example, the GRI may be calculated according to Equation 1 below:

GRI=(3.0×VLow)+(2.4×Low)+(1.6×VHigh)+(0.8×High) [Equation 1]

In Equation 1, ‘VLow’ and ‘Low’ are elements of the hypoglycemia risk index, respectively. ‘VLow’ means a ratio of time during which blood glucose is in a state less than about 54 mg/dL, and ‘Low’ means a ratio of time during which the blood glucose is in a state equal to or greater than about 54 mg/dL and less than 70 mg/dL. Similarly, ‘VHigh’ and ‘High’ are elements of the hyperglycemia risk index, respectively. ‘VHigh’ means a ratio of time during which the blood glucose is in a state exceeding about 250 mg/dL, and ‘High’ means a ratio of time during which the blood glucose is in a state equal to or less than about 250 mg/dL and exceeding 180 mg/dL.

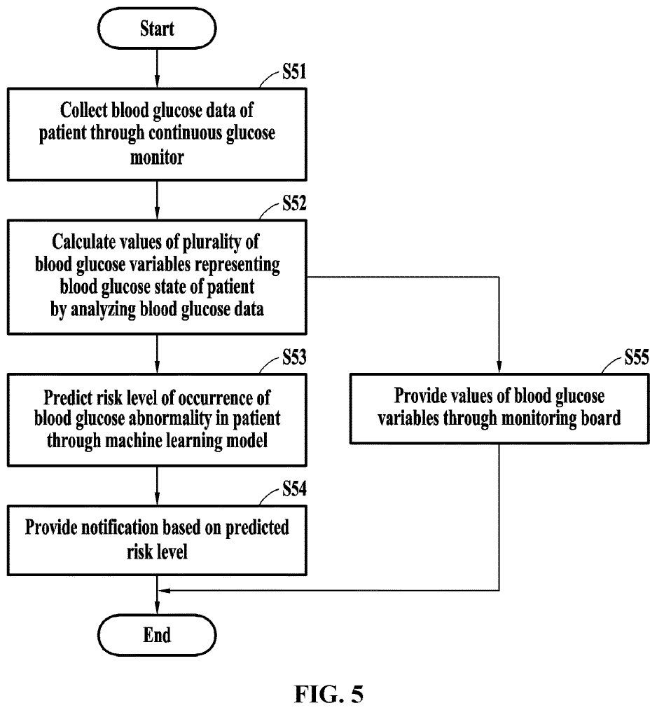

In addition, for example, the data analyzer 32 may early predict various information on a blood glucose abnormality by jointly analyzing blood glucose-related information and patient's medical information (e.g., clinical information, etc.) through a machine learning model. For this, descriptions of FIG. 5 and subsequent drawings may be referred to.

The medical information of the patient may include, for example, clinical information and non-clinical information. In addition, the clinical information may include, for example, a type of a patient (e.g., before surgery, after surgery, emergency patient, etc.), demographic information, diagnosis information (e.g., diagnosis name, etc.), prescription information, test results (e.g., blood test result, physical test result), surgery information, medication history, disease history, blood glucose-related information, and the like, but is not limited thereto. In addition, the non-clinical information may include, for example, administrative information, insurance information, and the like, but is not limited thereto.

In addition, for example, the data analyzer 32 may detect a blood glucose abnormality (e.g., rapid blood glucose fluctuation, etc.) of the patient in real time by analyzing the blood glucose data. The data analyzer 32 may detect the blood glucose abnormality using a machine learning model, and may also detect the blood glucose abnormality in another method.

Next, the data provider 33 is a module that provides blood glucose-related information (e.g., blood glucose data, blood glucose state information, etc.) of a patient. For example, the data provider 33 may provide various blood glucose-related information of the patient in real time or near real time through a monitoring board, and for this, descriptions of FIGS. 5 to 7E may be referred to.

Next, the notifier 34 is a module that provides a notification for blood glucose management to a patient and/or a medical staff. For example, when blood glucose abnormality information (e.g., risk level, etc.) predicted by the machine learning model satisfies a preset condition, the notifier 34 may provide a notification through the patient terminal 12. The notifier 34 may also provide such a notification to the medical staff. Alternatively, the notifier 34 may provide a notification to the patient and/or the medical staff when a blood glucose abnormality is detected in the patient. For a specific method of providing a notification, descriptions of FIG. 5 and the like may be referred to.

For reference, the term ‘notification’ may be used interchangeably with expressions such as ‘alarm’, ‘alert’, and ‘warning’ depending on the context.

For detailed operations of the blood glucose management system 10, descriptions of FIG. 5 and subsequent drawings may be further referred to.

The above-described blood glucose management system 10 or the components 31 to 34 thereof may be implemented as at least one computing device. For example, all functions of the blood glucose management system 10 may be implemented in one computing device, or a first function of the blood glucose management system 10 may be implemented in a first computing device and a second function thereof may be implemented in a second computing device. Alternatively, a specific function of the blood glucose management system 10 may be implemented in a plurality of computing devices.

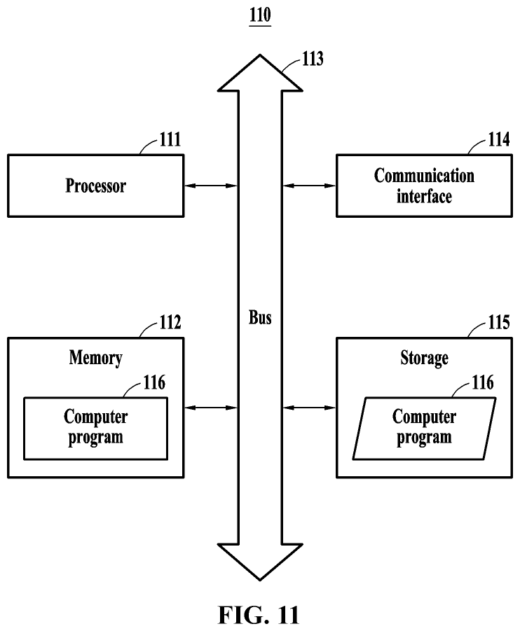

The computing device may include any device having a computing (or processing) function, and reference may be made to FIG. 11 for an example of such a device. Since the computing device is an aggregate in which various components (e.g., a memory, a processor, etc.) interact, it may be named a ‘computing system’ in some cases. Of course, the term “computing system” may also encompass a concept of an aggregate in which a plurality of computing devices interact.

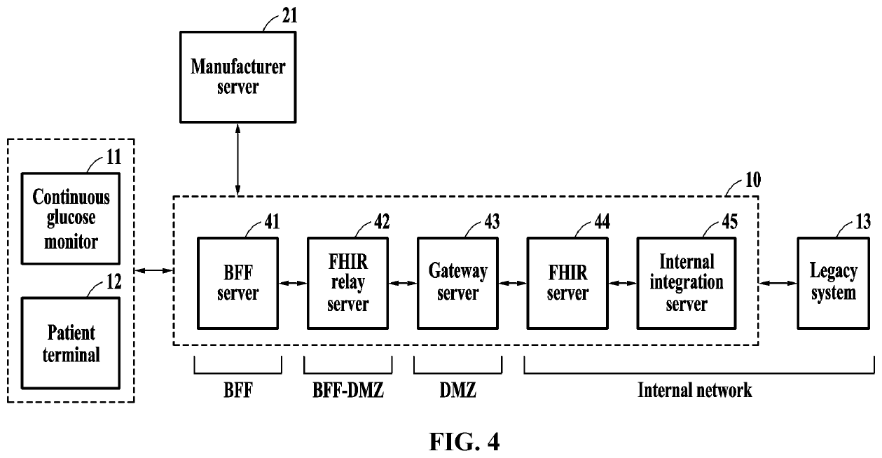

FIG. 4 is an exemplary view for describing an implementation example of the blood glucose management system 10 described above.

As shown in FIG. 4, the blood glucose management system 10 may include a Backend For Frontend (BFF) server 41, an FHIR relay server 42, a gateway server 43, an FHIR server 44, and an internal integration server 45. The modules 31 to 34 illustrated in FIG. 3 may be implemented in, for example, the internal integration server 45, but the scope of the present disclosure is not limited thereto. For example, the modules 31 to 34 may be distributed and implemented in different servers (e.g., 41 and 45, etc.).

As illustrated, the FHIR server 44, the internal integration server 45, and the legacy system 13 may be located in an internal network of a medical institution, and the gateway server 43 may be located in a Demilitarized Zone (DMZ) area for security of the internal network. Hereinafter, each server will be briefly described.

The BFF server 41 is a server responsible for an interface function with the patient terminal 12. For example, the BFF server 41 may receive and process a request from a healthcare app, installed in the patient terminal 12, through an API, and may transfer a specific request to the FHIR relay server 42. In addition, the BFF server 41 may receive health data (or a request including the health data) of a patient from the healthcare app, and may transfer the received health data to the FHIR relay server 42. The BFF server 41 may be understood as an app server linked with the healthcare app. Examples of the health data may include blood glucose data, activity data (e.g., data of activities related to blood glucose, such as exercise, meals, sleep, medication, and drug administration), and the like, but are not limited thereto.

Next, the FHIR relay server 42 is a server performing processing according to an FHIR protocol. The FHIR relay server 42 may be understood to internally play a role of an FHIR client. The FHIR relay server 42 may convert health data (or a request, etc.), received from the BFF server 41, according to the FHIR protocol, and transfer the converted health data to the gateway server 43. Of course, the FHIR relay server 42 may also transfer data (or a response to a request, etc.), received from the gateway server 43, to the BFF server 41.

Next, the gateway server 43 is a server playing a role of a gateway that mediates communication with the outside while maintaining security of an internal network. For example, the gateway server 43 may transfer the health data (i.e., the converted health data, the converted request, etc.), received from the FHIR relay server 42, to the FHIR server 44 located in the internal network.

Next, the FHIR server 44 is a server that reads and processes health data (or a request, etc.) converted according to the FHIR protocol. For example, the FHIR server 44 may transfer the health data (i.e., the converted health data, the converted request, etc.), received from the gateway server 43, to the internal integration server 45.

Finally, the internal integration server 45 is a server that integrally analyzes and manages health data, medical information/data, and the like of a patient. For example, the internal integration server 45 may receive medical information of the patient from the legacy system 13, and may also provide health data of the patient to the legacy system 13. In addition, the internal integration server 45 may receive the health data of the patient from the FHIR server 44, or acquire the health data from a storage of the FHIR server 44. In addition, the internal integration server 45 may provide the health data, the medical information, and the like of the patient to a medical staff through a monitoring board (e.g., the monitoring board may be provided through a UI of a legacy system). The internal integration server 45 may integrally analyze and manage data of a plurality of patients.

As described above, the implementation example of the blood glucose management system 10 described above has been described with reference to FIG. 4. Hereinafter, various methods that may be performed in the blood glucose management system 10 described above will be described with reference to FIG. 5 and subsequent drawings.

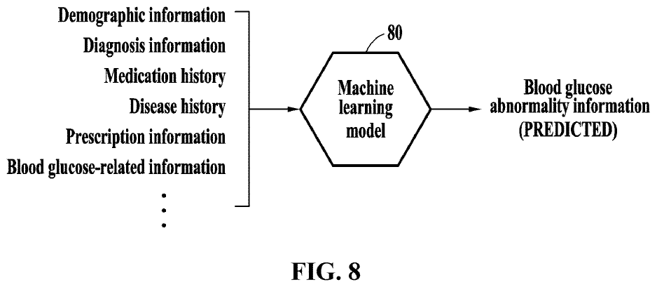

Hereinafter, for convenience of understanding, the description will be continued assuming that all steps/operations of methods to be described below are performed in the blood glucose management system 10 in the environment illustrated in FIG. 2. Therefore, when a subject of a specific step/operation is omitted, the corresponding step/operation may be understood to be performed by the blood glucose management system 10. However, in an actual environment, some steps/operations of the method to be described below may be performed by another computing device. For example, training of a machine learning model (refer to FIG. 8, etc.) for predicting a blood glucose abnormality may be performed by another computing device.

Hereinafter, for convenience of description, the blood glucose management system 10 will be abbreviated as a ‘system’.

FIG. 5 is an exemplary flowchart illustrating a blood glucose management method using a CGM according to some embodiments of the present disclosure. However, this is only an exemplary embodiment for achieving the purpose of the present disclosure, and it is obvious that some steps may be added or deleted as necessary.

As shown in FIG. 5, the blood glucose management method according to embodiments may start at step S51 of collecting blood glucose data of a patient through the CGM 11. For example, the system 10 (e.g., the data collector 31) may collect the blood glucose data of the patient in real time or near real time through an API provided by the manufacturer server 21.

In step S52, values of a plurality of blood glucose variables representing a blood glucose state of the patient are calculated by analyzing the blood glucose data. For example, the system 10 (e.g., the data analyzer 32) may calculate the values of the blood glucose variables, as illustrated in Table 1, by analyzing the blood glucose data (i.e., raw measurement data).

Steps S53 and S55, which will be described later, may be understood to be performed regardless of order.

In step S53, a risk level of an occurrence of a blood glucose abnormality in the patient is predicted through a machine learning model (i.e., a trained model). For example, the system 10 (e.g., the data analyzer 32) may predict the risk level (i.e., a probability) of the occurrence of the blood glucose abnormality in the patient within a specific time/period (e.g., 24 hours) from a current time point through the machine learning model (refer to FIG. 8). Examples of the blood glucose abnormality may include hypoglycemia, hyperglycemia, rapid blood glucose variability, an abnormal blood glucose pattern, and the like, but are not limited thereto.

In some cases, the system 10 may predict a time point (i.e., an expected time point) at which a blood glucose abnormality will occur, a type of the blood glucose abnormality, a cause of occurrence of the blood glucose abnormality, a duration of the blood glucose abnormality, and the like.

A structure, a training method, and the like of a machine learning model may be designed in various ways, which will be described later with reference to FIGS. 8 to 10.

In step S54, a notification is provided based on the predicted risk level. For example, when the predicted risk level satisfies a preset condition (e.g., equal to or greater than a threshold value), the system 10 (e.g., the notifier 34) may transmit a notification message to a patient and/or a medical staff (e.g., medical staff in charge of treating the patient). The notification message may be transmitted to the corresponding patient through, for example, a healthcare app installed in the patient terminal 12, but the scope of the present disclosure is not limited thereto.

The notification message may include, for example, a type of the blood glucose abnormality, a risk level thereof, an expected occurrence time point thereof, a recommended action or a blood glucose management guideline, a reason for occurrence thereof, an expected duration thereof, and the like, but is not limited thereto.

A specific method of providing the notification may vary depending on embodiments.

In some embodiments, the system 10 may immediately provide the notification when the predicted risk level is equal to or greater than the threshold value.

In some other embodiments, when the predicted risk level is equal to or greater than the threshold value, the system 10 may predict a duration of the blood glucose abnormality through the machine learning model. Then, when the predicted duration is equal to or greater than a reference value, the system 10 may immediately provide the notification. In a reverse case, the system 10 may withhold providing the notification.

In still other embodiments, when the predicted risk level is equal to or greater than the threshold value, the system 10 may withhold providing the notification until a standby timer expires, and monitor a blood glucose-related event (e.g., a meal, exercise, sleep, drug administration, etc.) of the patient. In addition, when an event increasing a blood glucose risk (i.e., an occurrence risk of a blood glucose abnormality) is detected before the standby timer expires, the system 10 may immediately provide the notification. Conversely, when an event decreasing the blood glucose risk is detected, the system 10 may reset the standby timer and predict the risk level again through the machine learning model (i.e., predicting again based on newly collected blood glucose data, etc.). Such a method may reduce inconvenience of the patient due to unnecessarily frequent notifications, and may also contribute to solving a problem in which a notification effect is degraded. For reference, even the same event may be determined as an event increasing the blood glucose risk or an event decreasing the blood glucose risk according to a state of the patient (e.g., if the patient is in a fasting state, exercise may be determined as an event increasing a hypoglycemia risk). In addition, an expiration time of the standby timer may be set dynamically. For example, the expiration time may be dynamically set according to the predicted risk level, the predicted occurrence time point, and the like (e.g., when the predicted risk level is low and the predicted occurrence time point is relatively far, the expiration time may be set to be longer).

In still other embodiments, when a predicted risk level is equal to or greater than a threshold value, the system 10 may predict an occurrence time point of a blood glucose abnormality through the machine learning model. In addition, when a difference between a current time point and the predicted occurrence time point is less than a reference value, the system 10 may immediately provide a notification. If the difference is equal to or greater than the reference value, the system 10 may withhold providing the notification until a standby timer expires, and monitor a blood glucose-related event (e.g., a meal, exercise, sleep, drug administration, etc.) of a patient. Then, when an event decreasing a blood glucose risk (i.e., an occurrence risk of the blood glucose abnormality) (e.g., exercise, drug administration, etc.) is detected, the system 10 may predict a risk level again through the machine learning model. Conversely, if an event increasing the blood glucose risk (e.g., a hyperglycemia meal, non-administration of a drug at a predetermined time, fasting exercise, etc.) is detected, the system 10 may immediately provide the notification.

Meanwhile, in some embodiments, the system 10 may detect the blood glucose abnormality of the patient in real time or near real time by analyzing blood glucose data. A specific method of detecting the blood glucose abnormality may be any method. In addition, the system 10 may transmit an immediate notification message to the patient (and/or medical staff) when the blood glucose abnormality is detected. At this time, the notification message may include information related to the blood glucose abnormality; and a patient-customized blood glucose management guideline (e.g., insulin administration adjustment, a diet improvement guideline, exercise therapy, etc.).

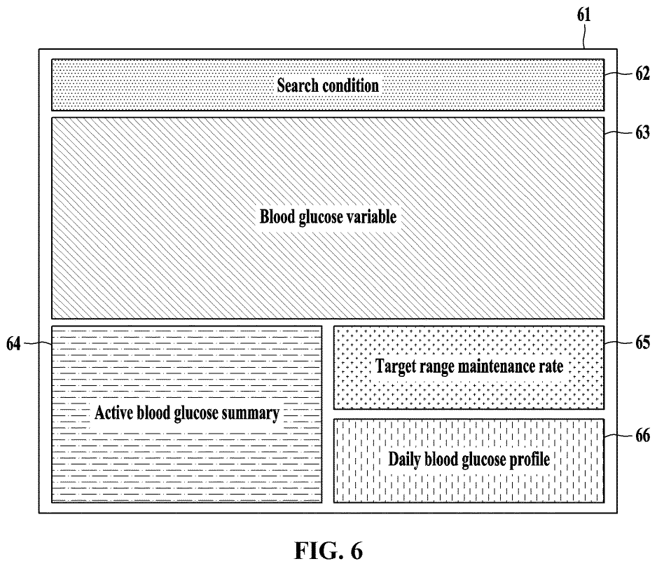

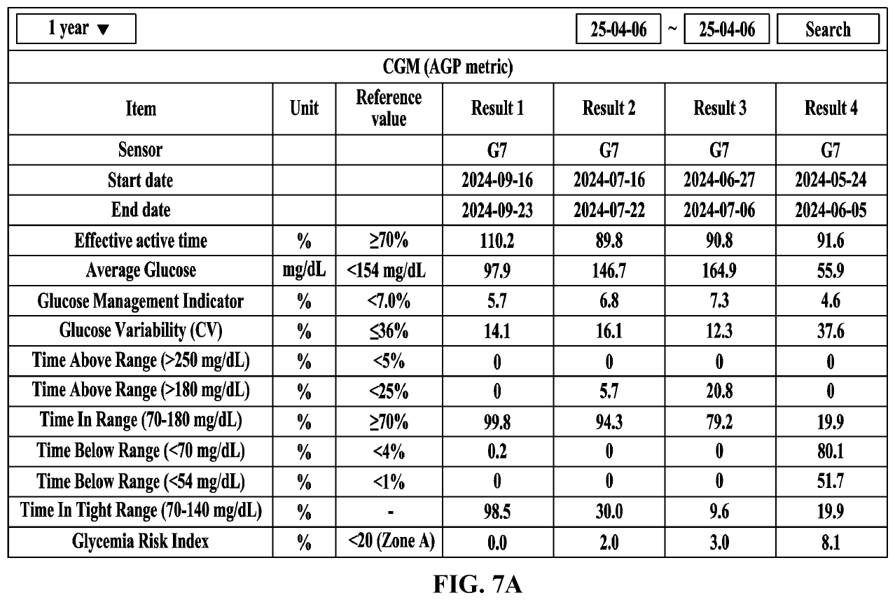

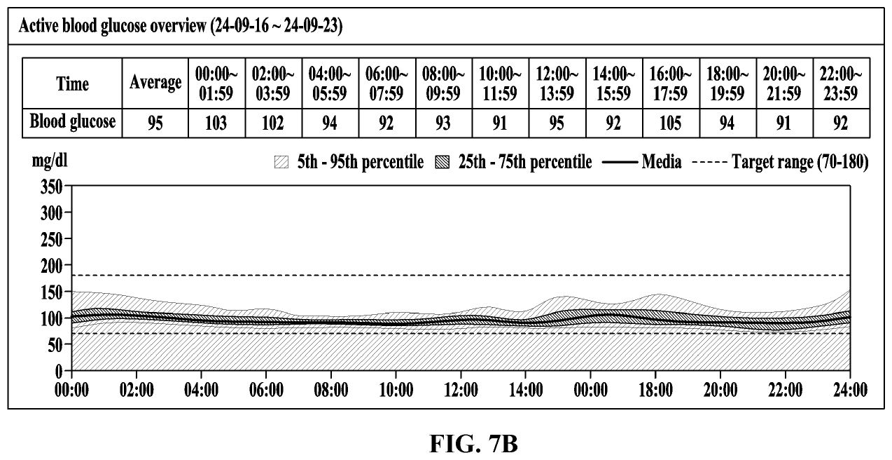

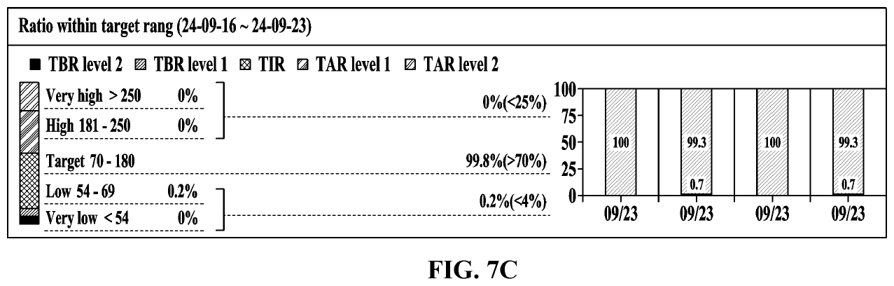

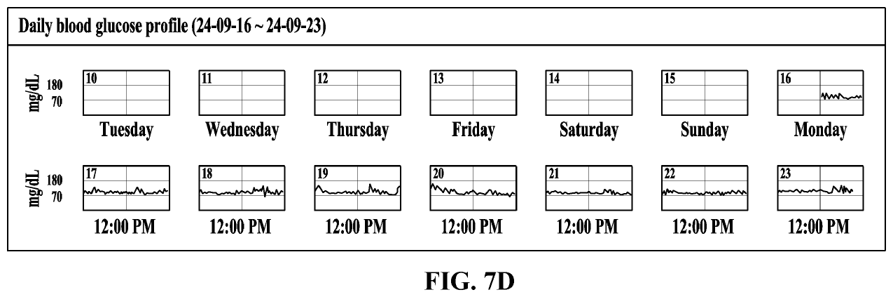

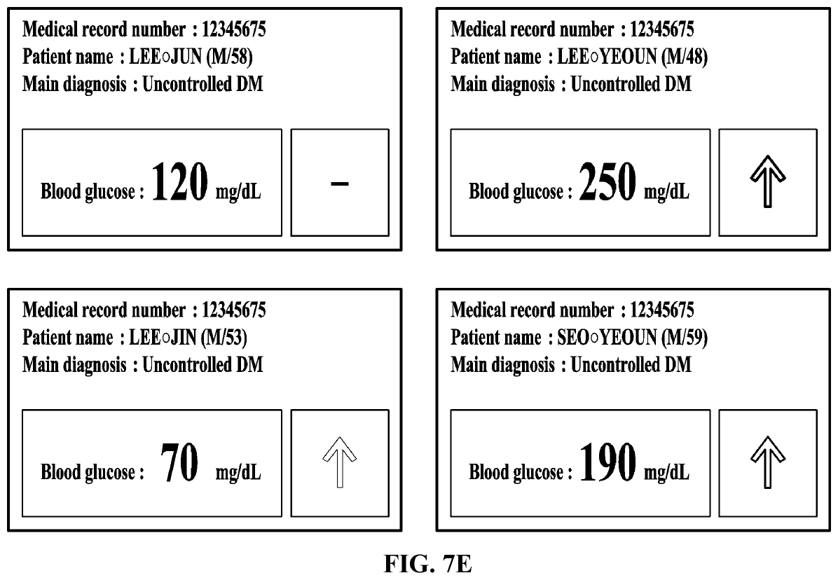

In step S55, values of blood glucose variables are provided through a monitoring board. For example, the system 10 (e.g., the data provider 34) may provide blood glucose-related information (e.g., blood glucose data, the values of the blood glucose variables, etc.) of the patient through a monitoring board (see FIGS. 6 to 7E). The system 10 may integrally provide blood glucose-related information of a plurality of patients through the monitoring board.

The monitoring board may be named, for example, a ‘monitoring interface’, a ‘monitoring screen’, a ‘monitoring User Interface (UI)’, a ‘monitoring Graphical User Interface (GUI)’, a ‘dashboard’, or the like in some cases.

The monitoring board may be designed in various forms, and a specific form thereof may vary depending on embodiments. Hereinafter, a design example of the monitoring board will be described with reference to FIGS. 6 to 7E so as to provide convenience of understanding.

FIG. 6 is an exemplary view for describing a screen configuration of a monitoring board 61 according to some embodiments of the present disclosure.

As shown in FIG. 6, the monitoring board 61 according to embodiments may include a plurality of interfaces (or areas) 62 to 66.

The first interface 62 is an interface for receiving an input of a search condition. The search condition may be, for example, a period, a patient name, a blood glucose variable, a hospital room number, or the like, but is not limited thereto. IG. 7A illustrates an implementation example of the first interface 62 and the second interface 63, and exemplifies a case where the first interface 62 is implemented to receive an input of period information as a search condition (see a ‘period input part’).

Information/data satisfying the search condition may be displayed on the second to fifth interfaces 63 to 66.

Next, the second interface 63 is an interface for displaying values of blood glucose variables. That is, the second interface 63 may be configured to display values of various blood glucose variables satisfying a search condition. FIG. 7A exemplifies a case where the second interface 62 is implemented to display the values of the blood glucose variables as shown in Table 1.

Next, the third interface 64 is an interface for displaying summary information on active blood glucose (e.g., a blood glucose change according to an activity, etc.). For example, the third interface 64 may be configured to display blood glucose-related information of a patient by time section or visualize a blood glucose change trend over time. In this case, a blood glucose change during a time period when the patient is highly active (e.g., working hours, exercise time, etc.) and an inactive time period (e.g., sleeping time), a blood glucose change pattern according to the activity, and the like may be intuitively conveyed. Reference may be made to FIG. 7B for an implementation example of the third interface 64. FIG. 7B exemplifies a case where the third interface 64 is implemented to display an average blood glucose for each time section and a blood glucose change trend over time (see a ‘blood glucose graph’).

Next, the fourth interface 65 is an interface for displaying a ratio of time during which the blood glucose of the patient is maintained within a target blood glucose range. Here, the target blood glucose range may be set to a range of a TITR, but the scope of the present disclosure is not limited thereto. The target blood glucose range may be set differently depending on a type of the patient (e.g., before surgery, recovery after surgery, emergency, etc.). Reference may be made to FIG. 7C for an implementation example of the fourth interface 65. FIG. 7C exemplifies a case where the fourth interface 65 is implemented to display a ratio of time during which the blood glucose of the patient belongs to each range (e.g., the target blood glucose range, a hypoglycemia range, a hyperglycemia range, etc.) on a daily basis.

Finally, the fifth interface 66 is an interface for displaying a blood glucose profile of the patient. Here, the blood glucose profile means blood glucose pattern information that visually or numerically summarizes a change tendency by time zone, a highest/lowest blood glucose, blood glucose stability, a postprandial blood glucose response, and the like based on blood glucose data measured over a predetermined period (e.g., a day, a week, etc.). For example, the fifth interface 66 may be configured to display a daily blood glucose profile of the patient, and reference may be made to FIG. 7D for an implementation example thereof. FIG. 7D exemplifies a case where the fifth interface 66 is implemented to visualize the daily blood glucose profile of the patient in a graph form.

FIG. 7E illustrates an implementation example of a monitoring board 71 (or a specific interface of the board) integrally providing blood glucose management of a plurality of patients. FIG. 7E assumes a case in which the monitoring board 71 visually provides blood glucose changes of inpatients and is utilized by medical staff of an intensive care unit or a ward.

As shown in FIG. 7E, the monitoring board 71 may highlight (refer to ‘color’), summarize and display a current blood glucose value, a blood glucose state (e.g., hyperglycemia, hypoglycemia, a normal blood glucose within a target range), a change aspect, and the like so that blood glucose changes of several patients may be intuitively recognized. In addition, the monitoring board 71 may be configured such that detailed blood glucose information of a specific patient is displayed when a blood glucose summary information area 72 of the patient is selected. Here, the detailed blood glucose information may mean, for example, information provided through at least one of the interfaces 62 to 66 described with reference to FIGS. 6 to 7D, but is not limited thereto. For example, the detailed blood glucose information may include a ratio of time during which the blood glucose of the patient is maintained within the target blood glucose range, information on active blood glucose, and the like, and may be provided to inquire about statistics of the day, 1 week, 2 weeks, 4 weeks, and the like.

As described above, the monitoring board 61 according to some embodiments of the present disclosure has been described with reference to FIGS. 6 to 7E. The above-described monitoring board 61 may be utilized to effectively monitor and manage the blood glucose state of the patient by providing blood glucose-related information in real time or near real time, and may also be utilized for integral blood glucose state management for a plurality of patients.

Hereinafter, an exemplary structure, a training method, and the like of a machine learning model 80 used for predicting a blood glucose abnormality will be described with reference to FIGS. 8 to 10.

FIG. 8 exemplifies input/output data of the machine learning model 80 according to some embodiments of the present disclosure.

As shown in FIG. 8, the machine learning model 80 may be configured to receive clinical information (or medical information), such as demographic information, diagnosis information, prescription information, medication history, and disease history, and blood glucose-related information of a patient as an input, and early predict and output blood glucose abnormality information. That is, the machine learning model 80 may be configured to predict information on a blood glucose abnormality in advance by comprehensively analyzing the clinical information and the blood glucose-related information of the patient. In this way, when even the clinical information of an individual patient is considered, the prediction on the blood glucose abnormality may be performed in a more precise and personalized form.

As described above, the blood glucose-related information may include blood glucose data (i.e., raw measurement data), and blood glucose state information composed of values of blood glucose variables. Hereinafter, for convenience of understanding, the description will be continued assuming a case in which the blood glucose-related information is composed of the values of the ‘blood glucose variables’.

The blood glucose abnormality information may include, for example, a type of the blood glucose abnormality (e.g., hypoglycemia, hyperglycemia, rapid blood glucose fluctuation, abnormal blood glucose pattern, etc.), whether the blood glucose abnormality occurs, an expected occurrence time point, a duration of blood glucose abnormality, a reason for occurrence thereof, or a combination thereof, but is not limited thereto.

The machine learning model 80 may be designed and implemented in various ways. For example, the machine learning model 80 may be designed and implemented based on various types of models/algorithms such as a decision tree, a random forest, an eXtreme Gradient Boosting (XGBoost), a Light Gradient Boosting Machine (LightGBM), a Categorical Boosting (CatBoost), a Support Vector Machine (SVM), a logistic regression, an Adaptive Boost (AdaBoost), and a neural network, and a specific type/form/structure and the like thereof may vary depending on a task of the machine learning model 80. Those skilled in the art will already be familiar with a basic structure, a training method, and the like of the exemplified models, and thus a description thereof will be omitted.

The above-described machine learning model 80 may be trained in a supervised learning method using a dataset of normal persons and patients. Those skilled in the art will already be familiar with a meaning of training being performed in the supervised learning method, and thus a description thereof will be omitted. For reference, details of a label used for the supervised learning may vary depending on the task of the machine learning model 80.

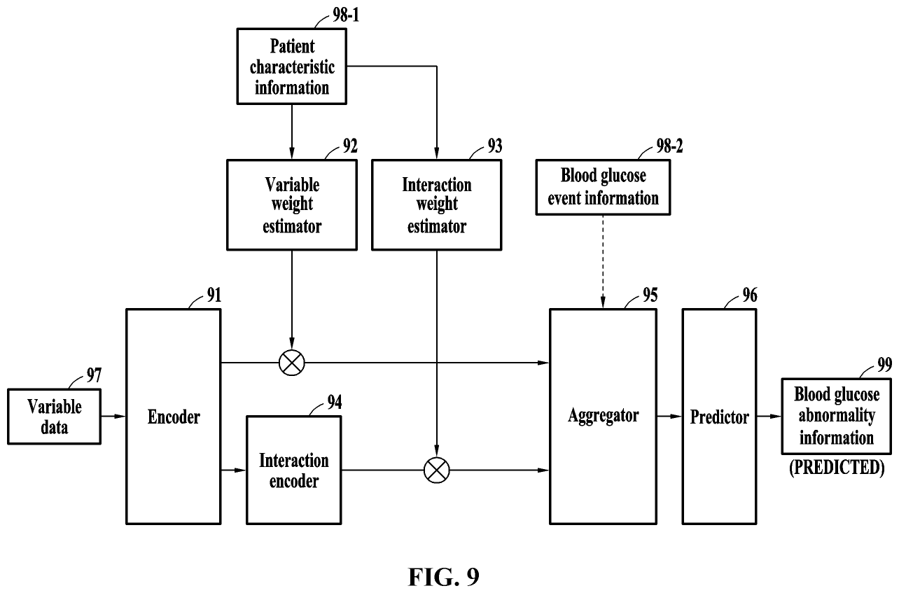

FIG. 9 is a view for explaining an exemplary structure and operation of the machine learning model 80 according to some embodiments of the present disclosure. FIG. 9 illustrates a structure of the machine learning model 80 designed to perform a prediction specialized for an individual patient, and assumes a case in which the model 80 is implemented based on the neural network.

As shown in FIG. 9, in some embodiments, the machine learning model 80 may include an encoder 91, a variable weight estimator 92, an interaction weight estimator 93, an interaction encoder 94, an aggregator 95, and a predictor 96.

The encoder 91 is a neural network module that generates an embedding by encoding variable data 97. Here, the variable data 97 may be composed of values of various blood glucose variables, and may correspond to time-series data. For example, the encoder 91 may be configured to receive a value of each of the blood glucose variables as an input and generate a variable embedding (e.g., an embedding vector) corresponding to each of the values.

The encoder 91 may be implemented as any type/form of a neural network. For example, the encoder 91 may be implemented based on a Multi-Layer Perceptron (MLP) (or a fully-connected layer), and may be implemented as another form of a neural network.

Next, the variable weight estimator 92 is a module that estimates a weight assigned to each of the blood glucose variables based on characteristic information 98-1 of a patient. Here, the characteristic information 98-1 may include clinical information of the patient, and may further include other characteristic information. In addition, the characteristic information 98-1 may be encoded in an appropriate form and input to the variable weight estimator 92. For example, the variable weight estimator 92 may be configured to estimate and output a weight (i.e., a variable weight) of each of the blood glucose variables specialized for an individual patient by analyzing the clinical information 98-1 of the patient. Such a variable weight may be understood as a factor for reflecting the point that a degree of influence of each blood glucose variable on a blood glucose abnormality may differ for each patient.

In some cases, the variable weight estimator 92 may be configured to receive a representative vector (e.g., a center vector, a weighted average vector, etc.) of a cluster to which the characteristic information 98-1 of the patient belongs, and such technical contents may also be applied to the interaction weight estimator 93.

The variable weight estimator 92 may be implemented in any form. For example, the variable weight estimator 92 may be implemented based on a neural network module such as an MLP, and may also be implemented as a module that performs an attention operation.

Next, the interaction weight estimator 93 is a module that estimates an interaction weight assigned to predefined blood glucose variable pairs based on the characteristic information 98-1 of the patient. For example, the interaction weight estimator 93 may be configured to estimate and output a weight (i.e., an interaction weight) of each of the blood glucose variable pairs specialized for an individual patient by analyzing the clinical information 98-1 of the patient. Such an interaction weight may be understood as a factor for reflecting the point that a degree of influence of an interaction between the blood glucose variables on the blood glucose abnormality may differ for each patient. The interaction weight estimator 93 may be implemented in a manner similar to the variable weight estimator 92.

Next, the interaction encoder 94 is a module that generates an interaction embedding corresponding to each of the blood glucose variable pairs. For example, the interaction encoder 94 may be configured to generate an interaction embedding corresponding to each of the predefined blood glucose variable pairs based on variable embeddings output by the encoder 91.

The interaction encoder 94 may be implemented in any form. For example, the interaction encoder 94 may be implemented based on a neural network module such as an MLP, and may be implemented as a module performing operations such as an average, a concatenation, and an elementwise multiplication on two variable embeddings.

Next, the aggregator 95 is a module generating an integrated embedding by aggregating variable embeddings, in which a weight is reflected, and interaction embeddings. In other words, the aggregator 95 may be configured to generate the integrated embedding by aggregating variable embeddings, in which a variable weight is reflected, and interaction embeddings in which an interaction weight is reflected.

The aggregator 95 may be implemented in any form. For example, the aggregator 95 may be implemented based on a neural network module such as an MLP, and may be implemented in another method.

In some embodiments, the aggregator 95 may be configured to further receive an input of blood glucose-related event information 98-2 of a patient, as illustrated in the drawing. The blood glucose-related event information 98-2 may be encoded in an appropriate method and input to the aggregator 95. In this case, the predictor 96 may predict blood glucose abnormality information 99 by considering even a blood glucose-related event (e.g., exercise, sleep, a meal, drug administration, etc.) of the patient.

Finally, the predictor 96 is a neural network module predicting the blood glucose abnormality information 99 based on the integrated embedding. That is, the predictor 96 may be configured to predict blood glucose abnormality information 99 of an individual patient by comprehensively analyzing various information (e.g., values of blood glucose variables and various clinical information) contained in the integrated embedding. The predictor 96 may be implemented based on, for example, an MLP, but the scope of the present disclosure is not limited thereto.

The predictor 96 may be configured to output a risk level (i.e., a probability) of an occurrence of the blood glucose abnormality in the patient within (or after) a specific time/period (e.g., 6 hours, 12 hours, 1 day, 3 days, etc.) (e.g., configured to output a confidence score on whether the blood glucose abnormality occurs), and may be configured to output a type of the blood glucose abnormality (e.g., hypoglycemia, hyperglycemia, etc.), a cause of occurrence thereof, an expected occurrence time point thereof, a duration thereof, and the like (e.g., configured to output a confidence score for each cause of occurrence).

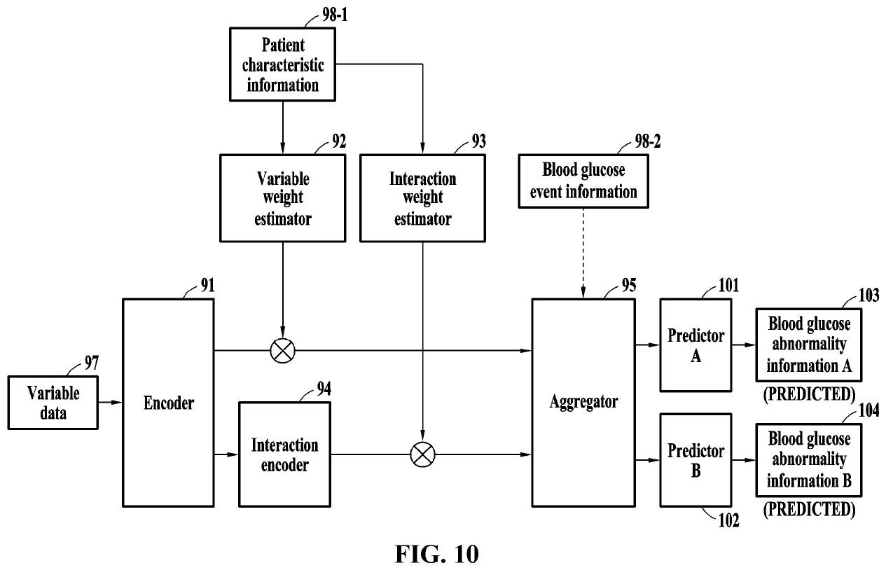

In some embodiments, the machine learning model 80 more accurately predicting the blood glucose abnormality information through a multi-task learning technique may be built. Hereinafter, such embodiments will be described with reference to FIG. 10.

FIG. 10 is an exemplary view for describing a multi-task learning-based model training method according to some embodiments of the present disclosure. Hereinafter, for clarity of the present disclosure, names such as ‘predictor A’ and ‘predictor B’ will be named as a ‘first predictor’ and a ‘second predictor’, and such a naming rule will be equally applied to other items in which similar names are used.

As shown in FIG. 10, the system 10 may perform multi-task learning using a plurality of predictors (e.g., 101 and 102) connected to the machine learning model 80. FIG. 10 exemplifies a case in which two predictors 101 and 102 are connected to the aggregator 95 of the machine learning model 80, but the scope of the present disclosure is not limited thereto. For example, a third predictor (not illustrated) may be further connected to the aggregator 95. At least one of the predictors 101 and 102 may mean the predictor 96 of FIG. 9 performing a target task.

The first predictor 101 is a neural network module predicting first blood glucose abnormality information 103 (hereinafter, abbreviated as ‘first information’), and the second predictor 102 is a neural network module predicting second blood glucose abnormality information 104 (hereinafter, abbreviated as ‘second information’) different from the first information (for an example of each information, see below). That is, the first predictor 101 and the second predictor 102 may be configured to perform different tasks (i.e., a task of predicting the first information and a task of predicting the second information) based on an integrated embedding.

The types and number of tasks for the multi-task learning may be variously defined and designed.

For example, the machine learning model 80 may be trained through a first task of predicting a risk level of an occurrence of a blood glucose abnormality within a specific time; and a second task of predicting a cause of the occurrence of the blood glucose abnormality (i.e., the first information 103 is the risk level of the occurrence of the blood glucose abnormality within the specific time, and the second information is the cause of the occurrence of the blood glucose abnormality). That is, parameters of the first predictor 101, the aggregator 95, the encoders 91 and 94, and/or the weight estimators 92 and 93 may be updated based on a prediction loss of the first task, and parameters of the second predictor 102, the aggregator 95, the encoders 91 and 94, and/or the weight estimators 92 and 93 may be updated based on a prediction loss of the second task.

As another example, the machine learning model 80 may be trained through a first task of predicting a risk level of an occurrence of the blood glucose abnormality within a first time; and a second task of predicting a risk level of an occurrence of the blood glucose abnormality within a second time different from the first time (i.e., the first information is the risk level of the occurrence of the blood glucose abnormality within the first time, and the second information is the risk level of the occurrence of the blood glucose abnormality within the second time). As a more specific example, the machine learning model 80 may be trained by performing a first task of predicting a risk level of an occurrence of the blood glucose abnormality within 24 hours through the first predictor 101, and performing a second task of predicting a risk level of an occurrence of the blood glucose abnormality within 48 hours through the second predictor 102.

As another example, the machine learning model 80 may be trained through a first task of predicting a risk level of an occurrence of a blood glucose abnormality within a specific time; and a second task of predicting an occurrence time point of the blood glucose abnormality (that is, the risk level of the occurrence of the blood glucose abnormality within the specific time is first information, and an expected occurrence time point of the blood glucose abnormality is second information). As a more specific example, the machine learning model 80 may be trained by performing a first task of predicting a risk level of an occurrence of a blood glucose abnormality within 24 hours through the first predictor 101; and performing a second task of predicting a time point at which the blood glucose abnormality will occur through the second predictor 102 corresponding to a regression head.

As another example, the machine learning model 80 may be trained through a first task of predicting a risk level of an occurrence of a blood glucose abnormality within a specific time; and a second task of predicting the number (or a duration, etc.) of occurrences of the blood glucose abnormality during a specific time (that is, the risk level of the occurrence of the blood glucose abnormality within the specific time is first information, and the expected number of occurrences of the blood glucose abnormality during the specific time is second information).

As another example, the machine learning model 80 may be trained using a task of predicting an accumulated blood glucose abnormality time during a specific time (or period), a task of predicting a blood glucose value after a specific time, a task of predicting a blood glucose-related event influencing blood glucose data, and the like.

As another example, the machine learning model 80 may be trained based on various combinations of the above-described examples.

When the training of the machine learning model 80 is completed, the system 10 may predict blood glucose abnormality information of a patient and provide a notification using the same. At this time, the blood glucose abnormality information predicted through the machine learning model 80 may be used to determine whether to provide a notification, a notification method, a content of a notification message, and the like. For example, a risk level of an occurrence of a blood glucose abnormality, an expected occurrence time point thereof, a duration thereof, and the like may be used to determine whether to provide a notification, a notification method, and/or a blood glucose management guideline to be included in a notification message.

In some embodiments, the system 10 may derive a patient-customized blood glucose management guideline through the machine learning model 80. Specifically, when a risk level of an occurrence of a blood glucose abnormality is predicted to be equal to or greater than a threshold value, the system 10 may repeatedly perform a risk level prediction while changing the blood glucose-related event information 98-2. Then, the system 10 may determine a patient-customized blood glucose management guideline based on the blood glucose-related event information 98-2 that has decreased a predicted risk level the most. For example, if the predicted risk level is decreased the most in a case in which a blood glucose-related event is ‘exercise’, the system 10 may determine the blood glucose management guideline of the patient as ‘exercise’.

As described above, the input/output data, exemplary detailed structure, training method, and the like of the machine learning model 80 according to some embodiments of the present disclosure have been described with reference to FIGS. 8 to 10.

As described above, the blood glucose management method using the CGM according to some embodiments of the present disclosure has been described with reference to FIGS. 5 to 10. According to the above description, blood glucose data of a patient may be collected in real time or near real time through the CGM 11, and values of various blood glucose variables representing a blood glucose state of the patient may be calculated by analyzing the collected blood glucose data. The values of the blood glucose variables may be provided to the patient and/or medical staff and the like through the monitoring board 61, and as a result, blood glucose states of patients may be thoroughly monitored and managed. For example, even blood glucose states of non-hospitalized patients (e.g., a patient recovering at home after surgery, a pre-hospitalization patient awaiting surgery, a discharged patient, etc.) may be effectively managed.

In addition, by acquiring blood glucose data of a patient through an API provided by the manufacturer server 21 of the CGM 11, the blood glucose data of the patient may be stably collected even in an environment in which direct interworking with the CGM 11 is limited.

In addition, as the system 10 is configured to perform processing (e.g., analysis, monitoring, etc.) on the blood glucose data independently of the legacy system 13 of a medical institution, blood glucose-related information of the patient may be reflected (or integrated) into the legacy system 13 without affecting a structure or performance of the legacy system 13.

In addition, by comprehensively analyzing the blood glucose-related information (e.g., the blood glucose data, values of blood glucose variables, etc.) and clinical information of the patient through the machine learning model 80, a risk level of an occurrence of a blood glucose abnormality in the patient may be accurately predicted. In addition, when the risk level is equal to or greater than a threshold value, a situation in which the blood glucose abnormality occurs in the patient may be prevented in advance by providing a notification to the patient and/or medical staff.

Hereinafter, an exemplary computing device 110 capable of implementing the above-described system 10 will be described with reference to FIG. 11.

FIG. 11 is an exemplary hardware configuration diagram illustrating a computing device 110.

As illustrated in FIG. 11, the computing device 110 may include at least one processor 111, a bus 113, a communication interface 114, a memory 112 loading a computer program 116 executed by the processor 111, and a storage 115 storing the computer program 116. However, only components related to the embodiments of the present disclosure are illustrated in FIG. 11. Accordingly, those skilled in the art to which the present disclosure pertains will appreciate that other general-purpose components may be further included in addition to the components illustrated in FIG. 11. That is, the computing device 110 may further include various components in addition to the components illustrated in FIG. 11. In addition, in some cases, the computing device 110 may be configured in a form in which some of the components illustrated in FIG. 11 are omitted. Hereinafter, each component of the computing device 110 will be described.

The processor 111 may control overall operations of each component of the computing device 110. The processor 111 may include at least one of a Central Processing Unit (CPU), a Micro Processor Unit (MPU), a Micro Controller Unit (MCU), a Graphic Processing Unit (GPU), a Neural Processing Unit (NPU), a Tensor Processing Unit (TPU), a Vision Processing Unit (VPU), an Accelerated Processing Unit (APU), and any type of processor well-known in the technical field of the present disclosure. In addition, the processor 111 may perform an operation on at least one application or program for executing specific operations/steps/methods. The computing device 110 may include at least one processor.

Next, the memory 112 may store various data, instructions, and/or information. The memory 112 may load the computer program 116 from the storage 115 to execute specific operations/steps/methods. The memory 112 may be implemented as a volatile memory such as a RAM, but the technical scope of the present disclosure is not limited thereto.

Next, the bus 113 may provide a communication function between the components of the computing device 110. The bus 113 may be implemented as various types of buses such as an address bus, a data bus, and a control bus.

Next, the communication interface 114 may support wired/wireless communication of the computing device 110. To this end, the communication interface 114 may be configured to include a communication module (e.g., an RF module supporting wireless communication) well-known in the technical field of the present disclosure, and may support various communication methods.

Next, the storage 115 may non-temporarily store one or more computer programs 116. The storage 115 may include a non-volatile memory such as a Read Only Memory (ROM), an Erasable Programmable ROM (EPROM), an Electrically Erasable Programmable ROM (EEPROM), or a flash memory, a hard disk, a removable disk, or any type of computer-readable recording medium well-known in the technical field to which the present disclosure pertains.

Next, the computer program 116 may include instructions that cause the processor 111 to perform specific operations/steps/methods when loaded into the memory 112. That is, the processor 111 may perform specific operations/steps/methods by executing the loaded instructions.

For example, the computer program 116 may include instructions for performing an operation of collecting blood glucose data measured through the CGM 11 worn by the patient; an operation of calculating values of a plurality of blood glucose variables representing a blood glucose state of the patient by analyzing the collected blood glucose data; and an operation of providing the values of the blood glucose variables through the monitoring board 61.

As another example, the computer program 116 may include instructions for performing at least some of the operations/steps/methods described with reference to FIGS. 1 to 10.

In the exemplified case, the system 10 according to some embodiments of the present disclosure may be implemented through the computing device 110.

In some embodiments, the computing device 110 illustrated in FIG. 11 may mean a virtual machine implemented based on cloud technology. For example, the computing device 110 may be a virtual machine operating in one or more physical servers included in a server farm. In this case, at least some of the processor 111, the memory 112, and the storage 115 illustrated in FIG. 11 may be virtual hardware, and the communication interface 114 may also be implemented as a virtualized networking element such as a virtual switch.

As described above, the exemplary computing device 110 capable of implementing the system 10 according to some embodiments of the present disclosure has been described with reference to FIG. 11.

As described above, various embodiments of the present disclosure and effects according to the embodiments have been mentioned with reference to FIGS. 1 to 11. Effects according to the technical spirit of the present disclosure are not limited to the effects mentioned above, and other effects not mentioned may be clearly understood by those skilled in the art from the following description.

According to some embodiments of the present disclosure, blood glucose data of a patient can be collected in real time or near real time through a CGM, and values of various blood glucose variables representing a blood glucose state of the patient can be calculated by analyzing the collected blood glucose data. The values of the blood glucose variables can be provided to the patient and/or medical staff and the like through a monitoring board, and as a result, blood glucose states of patients can be thoroughly monitored and managed. For example, even blood glucose states of non-hospitalized patients (e.g., a patient recovering at home after surgery, a pre-hospitalization patient awaiting surgery, a discharged patient, etc.) can be effectively managed.

In addition, by acquiring blood glucose data of a patient through an Application Programming Interface (API) provided by a manufacturer server of the CGM, the blood glucose data of the patient can be stably collected even in an environment in which direct interworking with the CGM is limited.

In addition, as a blood glucose management system is configured to perform processing (e.g., analysis, monitoring, etc.) on blood glucose data independently of a legacy system of a medical institution, blood glucose-related information of a patient can be reflected (or integrated) into the legacy system without affecting the structure or performance of the legacy system.

In addition, by comprehensively analyzing the blood glucose-related information (e.g., the blood glucose data, the values of the blood glucose variables, etc.) and clinical information of the patient through a machine learning model, a risk level of an occurrence of a blood glucose abnormality in the patient can be accurately predicted. Further, when the risk level is equal to or greater than a threshold value, a situation in which the blood glucose abnormality occurs in the patient can be prevented in advance by providing a notification to the patient and/or medical staff.

Furthermore, as the blood glucose management system is interlocked with the CGM and the legacy system of the medical institution and also functions as a platform for health management of the patient including blood glucose management, an integrated health and medical service can be provided to the patient.

Effects according to the technical spirit of the present disclosure are not limited to the effects mentioned above, and other effects not mentioned may be clearly understood by those skilled in the art from the following description.

In addition, even if it is described that a plurality of components are combined into one or operate in combination in the above embodiments, the technical spirit of the present disclosure is not necessarily limited to these embodiments. That is, within the scope of the purpose of the technical spirit of the present disclosure, all of the components may operate by being selectively combined into one or more.

The technical spirit of the present disclosure described above may be implemented as a computer-readable code on a computer-readable recording medium. The computer program recorded on the computer-readable recording medium may be transmitted to another computing device through a network such as the Internet and installed in the corresponding computing device, thereby being used in the corresponding computing device.

Although operations are shown in a specific order in the drawings, it should not be understood that the operations must necessarily be performed in the specific order shown in the drawings or in a sequential order, or that all the shown operations must be performed to obtain a desired result. In certain circumstances, multitasking and parallel processing may be advantageous. Although various embodiments of the present disclosure have been described with reference to the accompanying drawings, those of ordinary skill in the art to which the present disclosure pertains may understand that the technical spirit of the present disclosure may be implemented in other specific forms without changing the technical spirit or essential features thereof. Therefore, it should be understood that the embodiments described above are illustrative in all respects and not restrictive. The protection scope of the present disclosure should be construed by the following claims, and all technical spirits within the equivalent scope should be construed as being included in the scope of rights of the technical spirit defined by the present disclosure.

[Description of Symbols]

| | |
|---|---|
| 10: Blood glucose management system | 11: Continuous glucose monitor |
| 12: Patient terminal | 13: Legacy system |
| 21: manufacturer server | 31: Data collector |
| 32: Data analyzer | 33: Data provider |
| 34: Notifier | 41: BFF server |
| 42: FHIR relay server | 43: gateway server |
| 44: FHIR server | 45: internal integration server |
| 61, 71: monitoring board | 62, 63, 64, 65, 66: interface |
| 80: machine learning model | 91: Encoder |
| 92: Variable weight estimator | 93: Interaction weight estimator |
| 94: Interaction encoder | 95: Aggregator |
| 96, 101, 102: Predictor | |

What is claimed is:

1. A system for blood glucose management, the system comprising:
a data collector configured to collect blood glucose data of a patient, wherein the blood glucose data is measured through a continuous glucose monitor worn by the patient;
a data analyzer configured to analyze the collected blood glucose data to calculate values of a plurality of blood glucose variables representing a blood glucose state of the patient; and
a data provider configured to provide the values of the blood glucose variables through a monitoring board,
wherein the blood glucose management system is linked with a legacy system of a medical institution, and is configured to perform processing based on the blood glucose data independently of the legacy system.

2. The system according to claim 1, wherein the legacy system comprises an Electronic Medical Record (EMR) system.

3. The system according to claim 1, wherein the blood glucose management system provides at least some of the values of the blood glucose variables to the legacy system, and receives medical information of the patient from the legacy system.

4. The system according to claim 1, wherein the data collector collects blood glucose data of each of a plurality of patients, and the data provider integrally provides blood glucose-related information of the patients through the monitoring board.

5. The system according to claim 1, wherein the data collector collects the blood glucose data from a manufacturer server of the continuous glucose monitor through an Application Programming Interface (API).

6. The system according to claim 1, wherein the blood glucose variables comprise a Time In Tight Range (TITR),
wherein the TITR means a ratio of time during which a blood glucose is within a predetermined range,
wherein an upper limit of the predetermined range is set to a value between 130 mg/dL and 150 mg/dL, and
a lower limit of the predetermined range is set to a value between 60 mg/dL and 90 mg/dL.

7. The system according to claim 1, wherein the blood glucose variables comprise a Glycemia Risk Index (GRI),
wherein the GRI is calculated based on a hypoglycemia risk index and a hyperglycemia risk index,
wherein a higher weight is assigned to the hypoglycemia risk index than to the hyperglycemia risk index.

8. The system according to claim 1, wherein the data analyzer acquires clinical information of the patient from the legacy system, and predicts a risk level of an occurrence of a blood glucose abnormality in the patient within a specific time by jointly analyzing the collected blood glucose data and the clinical information through a machine learning model,
the clinical information comprises at least one of demographic information, diagnosis information, prescription information, a medication history, and a disease history of the patient, and
the blood glucose management system further comprises a notifier configured to provide a notification based on the predicted risk level.

9. The system according to claim 8, wherein the machine learning model comprises:
an encoder configured to generate variable embeddings corresponding to the blood glucose variables;
an interaction encoder configured to generate interaction embeddings corresponding to predefined blood glucose variable pairs based on the variable embeddings;
a variable weight estimator configured to estimate a variable weight, specialized for the patient, for each of the blood glucose variables based on the clinical information;
an interaction weight estimator configured to estimate an interaction weight, specialized for the patient, for each of the blood glucose variable pairs based on the clinical information;
an aggregator configured to generate an integrated embedding by aggregating the variable embeddings, in which the variable weight is reflected, and the interaction embeddings in which the interaction weight is reflected; and
a predictor configured to predict the risk level based on the integrated embedding.

10. The system according to claim 8, wherein the notifier:
withholds providing the notification until a preset standby timer expires when the predicted risk level is equal to or greater than a threshold value,
immediately provides the notification when an event increasing the occurrence risk of the blood glucose abnormality is detected in the patient before the standby timer expires, and
resets the standby timer and predicts the risk level again through the machine learning model, when an event decreasing the occurrence risk of the blood glucose abnormality is detected in the patient before the standby timer expires.

11. The system according to claim 1, wherein the monitoring board comprises:
a first interface displaying the values of the blood glucose variables;
a second interface displaying blood glucose-related information of the patient by time section;
a third interface displaying a ratio of time during which the blood glucose of the patient is maintained within a target blood glucose range; and
a fourth interface displaying a daily blood glucose profile of the patient.

12. The system according to claim 1, wherein the blood glucose management system comprises a Backend For Frontend (BFF) server, a Fast Healthcare Interoperability Resource (FHIR) relay server, a gateway server, an FHIR server, and an internal integration server,
the data collector, the data analyzer, and the data provider are implemented in the internal integration server,
the FHIR server and the internal integration server are located together with the legacy system in an internal network of the medical institution,
the BFF server is configured to transfer health data, received from a terminal of the patient, to the FHIR relay server,
the FHIR relay server is configured to convert the health data, received from the BFF server, according to an FHIR protocol and transfer the converted health data to the gateway server,
the gateway server is configured to transfer the converted health data to the FHIR server located in the internal network, and
the FHIR server is configured to transfer the converted health data to the internal integration server.

13. A method for blood glucose management using a continuous glucose monitor, the method being performed by at least one processor of a blood glucose management system and comprising:
collecting blood glucose data measured through the continuous glucose monitor worn by a patient;
calculating values of a plurality of blood glucose variables representing a blood glucose state of the patient by analyzing the collected blood glucose data; and
providing the values of the blood glucose variables through a monitoring board,
wherein the blood glucose management system is linked with a legacy system of a medical institution, and is configured to perform processing based on the blood glucose data independently of the legacy system.

14. The method according to claim 13, wherein the collecting of the blood glucose data comprises collecting the blood glucose data from a manufacturer server of the continuous glucose monitor through an Application Programming Interface (API).

15. The method according to claim 13, further comprising:
acquiring clinical information of the patient from the legacy system;
predicting a risk level of an occurrence of a blood glucose abnormality in the patient within a specific time by jointly analyzing the collected blood glucose data and the clinical information through a machine learning model; and
providing a notification based on the predicted risk level,
wherein the clinical information comprises at least one of demographic information, diagnosis information, prescription information, a medication history, and a disease history of the patient.

16. The method according to claim 13, wherein the monitoring board comprises:
a first interface displaying the values of the blood glucose variables;
a second interface displaying blood glucose-related information of the patient by time section;
a third interface displaying a ratio of time during which the blood glucose of the patient is maintained within a target blood glucose range; and
a fourth interface displaying a daily blood glucose profile of the patient.
